(19) United States
(12) Patent Application Publication
(10) Pub. No.: US 2005/0228433 A1
(43) Pub. Date: Oct. 13, 2005

(54) In situ implant and method of forming same

(76) Inventors: Weenna Bucay-Couto, Burlington, MA (US); Cliff Liu, Randolph, MA (US)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/801,231

(22) Filed: Mar. 16, 2004

(51) Int. Cl. A61M 29/00

(52) U.S. Cl. 606/200

(57) ABSTRACT

Methods of providing a polymeric body within a subject are described. The methods comprise: (a) injecting a fluid that comprises a crosslinkable polymer into a container (for instance, a balloon) that is positioned within the subject; (b) crosslinking the crosslinkable polymer in the container, thereby forming a crosslinked polymeric body in the container; (c) optionally washing the crosslinked polymeric body in the container; and (d) releasing the crosslinked polymeric body within the subject. Other aspects of the present invention are directed to devices for performing the above methods, and to the wide variety of polymeric bodies that can be formed using the above methods. Examples include antiadhesive bodies, aneurism fillers, embolic bodies, bulking agents, tissue scaffolding, drug delivery bodies, and so forth.

### FIELD OF THE INVENTION

The present invention relates to polymeric bodies, more particularly to polymeric bodies which are formed in situ by injecting polymeric compositions into the body of a patient and subsequently crosslinking them there.

### BACKGROUND OF THE INVENTION

Polymeric bodies, including polymeric implants, have significant potential for achieving beneficial effects in a wide variety of medical fields including tissue engineering, post-operative adhesion prevention, drug delivery and embolization, among many others.

Although polymeric bodies can be implanted (or inserted) as preformed solid bodies within a subject, it is also known to form polymeric bodies by injecting a fluid into the body, which fluid thereafter solidifies in situ.

Such systems have the characteristics of preformed bodies, for example, but are superior in several ways. For example, unlike preformed polymeric bodies, the fact that such systems solidify in situ means that they are able to conform to the contours of adjacent tissue in the body. Moreover, the ability to introduce the polymeric bodies in liquid form commonly enables the medical practitioner to resort to surgical techniques that are less invasive than those required for preformed implants—in some instances eliminating the need for surgery altogether.

### SUMMARY OF THE INVENTION

Various aspects of the present invention are directed to methods of providing a polymeric body within a subject. These methods comprise: (a) injecting a polymer containing fluid into a container that is positioned within the subject (e.g., an expandable container such as a balloon); (b) crosslinking the polymer, thereby forming a crosslinked polymeric body in the container (e.g., by injecting an ionic and/or covalent crosslinking agent into the container); (c) optionally, washing the crosslinked polymeric body in the container; and (d) releasing the crosslinked polymeric body within the subject.

Other aspects of the invention are directed to the wide variety of polymeric bodies that can be formed using the above methods. Examples include antiadhesive bodies, aneurism fillers, embolic bodies, bulking agents, tissue scaffolding, and drug delivery bodies, among others.

Other aspects of the present invention are directed to devices for providing a polymeric body within a subject. For example, according to one embodiment, a device is provided, which comprises: (a) a shaft comprising an inner lumen and (b) a balloon disposed at a distal end of the shaft, wherein the medical device is adapted to either (i) release the crosslinked polymeric body from the balloon and into the patient or (ii) to release both the balloon and the crosslinked body into the patient.

The present invention is advantageous, for example, in that the crosslinking process is conducted within the container, thereby reducing the exposure of the subject to undesirable materials, such as crosslinking agents, many of which are toxic. In addition, exposure to undesirable materials can be further reduced by washing the crosslinked polymeric body within the container prior to its release.

These and other embodiments and advantages of the present invention will become immediately apparent to those of ordinary skill in the art upon review of the Detailed Description and claims to follow.

### BRIEF DESCRIPTION OF THE DRAWINGS

FIGS. 1A-1C are schematic partial cross-sectional views, illustrating the in situ formation of a crosslinked polymeric body, in accordance with an embodiment of the present invention.

FIGS. 2 and 3 are schematic partial cross-sectional views illustrating two medical devices for the in situ formation of crosslinked polymeric bodies, in accordance with additional embodiments of the present invention.

### DETAILED DESCRIPTION OF THE INVENTION

One aspect of the present invention is directed to a novel method for providing a polymeric body within a subject, for example, a vertebrate subject, typically a mammalian subject, and more typically a human subject. The method comprises: (a) introducing a polymer containing fluid (for instance, a liquid or gel, typically a gel), which comprises a crosslinkable polymer, into a container that is positioned within the subject; (b) crosslinking the crosslinkable polymer in the container, thereby forming a crosslinked polymeric body; (c) optionally washing the crosslinked polymeric body in the container; and (d) releasing the crosslinked polymeric body within the subject.

As discussed in more detail below, a variety of polymers can be used in the methods of the present invention, including various biostable polymers and biodisintegrable polymers. Moreover, the polymers can be crosslinked by a variety of mechanisms, including various chemical crosslinking mechanisms.

Where chemical crosslinking is employed, a chemical crosslinking agent (typically an ionic crosslinking agent and/or a covalent crosslinking agent) can be introduced: (a) concurrently with the polymer-containing fluid, (b) prior to the introduction of the polymer-containing fluid, or (c) subsequent to the introduction of the polymer-containing fluid.

To some extent, the order of introduction will depend upon the kinetics of the crosslinking reaction. For example, if the crosslinking reaction proceeds quickly, it may be desirable to introduce the polymer-containing fluid into the container prior to the introduction of the crosslinking agent. As another example, if the crosslinking reaction proceeds slowly, it may be desirable to introduce the polymer-containing fluid concurrent with the crosslinking agent (including the introduction of a premixed blend of the polymer-containing fluid and the crosslinking agent) or after the introduction of the crosslinking agent.

In some embodiments, the polymeric body is “grown” by performing additional polymer introduction and crosslinking steps, with each cycle increasing the size of the crosslinked polymeric body. The growing body can be washed, for example, after each crosslinking step or after the final crosslinking step.

In some embodiments, the polymer-containing fluid is introduced as a mixture of fluids (e.g., two gels can be simultaneously introduced), or as a succession of fluids each with differing viscosities (e.g., a high viscosity gel, followed by a lower viscosity gel, and so forth). A crosslinking agent can be introduced after the introduction of each fluid or after multiple fluids have been introduced.

Beneficial containers for use in conjunction with the present invention include various expandable or flexible containers, which may have either elastic (e.g., an elastomer such as rubber) or inelastic (e.g., a metal foil) walls. Typically, the container is a balloon.

At the time of introduction into the subject, the container is typically attached to a delivery device, which has a lumen for injecting the polymer-containing fluid into the container once properly positioned into the body of a subject. The delivery device may also contain additional lumens, for example, for introduction of crosslinking agent, or for introduction or withdrawal of a washing fluid. In some embodiments, the device is a catheter, allowing for minimally invasive placement of the crosslinked body within the subject. In some embodiments, the device is an injection device in which the container is retracted within a sharp tipped sleeve (e.g., a large gauge needle) during advancement of the device to the location of interest, for instance, transdermal advancement.

It is noted that the crosslinked body may be introduced into the subject by essentially any route, including transdermal routes (including percutaneous routes) and transluminal routes (including transvascular, transrectal, transurethral, transvaginal, transnasal and transoral routes).

Once the polymeric body is crosslinked to the desired degree, it is optionally purified by washing the crosslinked body with a washing fluid, for example, water, saline, buffered solutions including buffered saline, Ringers solution, a non-toxic detergent solution, an organic solvent such as ethanol, mixtures of the same (e.g., water-diluted alcohol), or a progression of the same (e.g., washing with organic solvent(s), followed by physiological saline or a buffer). For this purpose, the delivery device may comprise separate lumens for introduction and withdrawal of the washing fluid as noted above. Washing is beneficial in many instances. For example, many known crosslinking agents, particularly covalent crosslinking agents, are slightly to highly toxic.

Ultimately, the crosslinked body is released from the delivery device. In some embodiments, the container, which has heretofore confined the polymer containing fluid and any crosslinking agent, is opened to release the crosslinked body. For example, where a balloon is used as the container, the balloon can be burst, cut, melted or otherwise opened to release the crosslinked body. In other embodiments, the container is severed from the delivery device, thereby releasing it, along with the crosslinked body, into the subject. For example, where a balloon is used as the container, it can be cut, melted (e.g., by heating the tip or the delivery device), or otherwise severed proximate the point where the balloon adjoins the delivery device. Where severed, the container may be formed, for example, from a biodisintegrable material.

Note that, in the present invention, the crosslinked body is substantially in its final three-dimensional form at the point of release from the container, with the crosslinked body experiencing no radical change in form during its release from the container. Hence, the present invention is readily distinguished from techniques in which previously crosslinked material is extruded from an outlet port of a container, which extrusion process would clearly result in a radical change in three-dimensional shape of the crosslinked material (e.g., with the material undergoing extreme deformation or flow), as it is forced from the container.

Because they are commonly formed in situ in containers that are expandable and flexible (e.g., balloons having either elastic or inelastic walls), the crosslinked bodies of the present invention typically reflect the shape of adjacent bodily tissue. For example, where the crosslinked body is formed in either a natural bodily cavity or lumen (e.g., a vascular, urinary, gastro-intestinal, nasal, vaginal, uterine, or bronchial cavity/lumen) or an artificially created bodily cavity/lumen (e.g. a surgical site created in tissue), sufficient polymer can be supplied such that the crosslinked body spans or even fills the cavity or lumen.

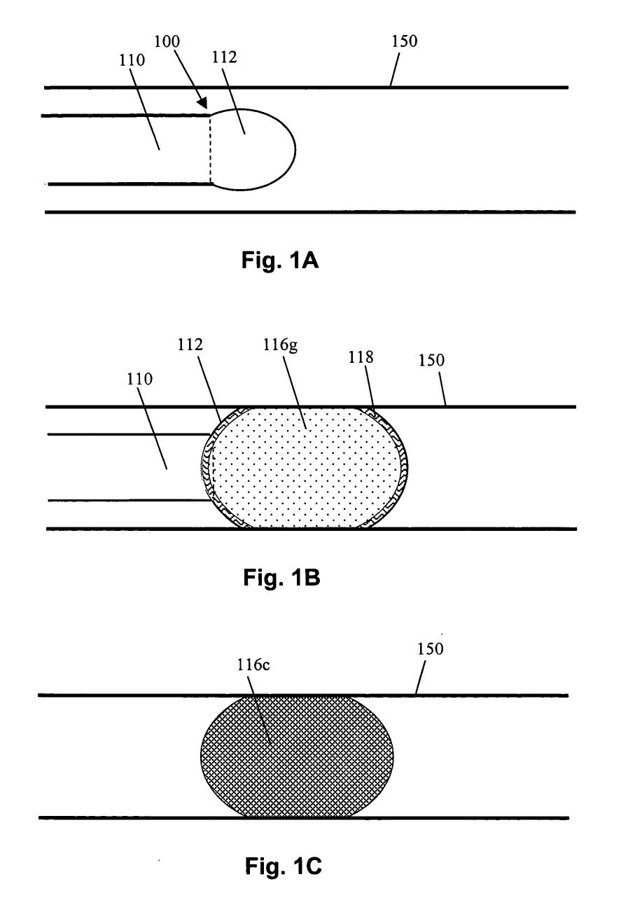

This is illustrated, in accordance with one embodiment of the invention, with reference to FIGS. 1A-1C. FIG. 1A shows a delivery device, in this instance a catheter 100 consisting of a hollow shaft 110 with a balloon 112 attached to its tip. After advancing the catheter 100 to a desired position in a bodily lumen 150 (e.g., an artery), the balloon 112 is filled, first with a polymer containing gel 116g, followed by a crosslinking agent 118, as seen in FIG. 1B. After washing the resulting crosslinked gel 116c to remove excess chemicals, the balloon is burst or cut, thereby releasing the crosslinked gel 116c within the bodily lumen 150 as shown in FIG. 1C.

In accordance with another specific embodiment, a first polymer containing gel, which also contains a therapeutic agent, is first injected inside the balloon. The polymer(s) within the first gel can be, for example, natural or synthetic, biostable or biodisintegrable, crosslinkable or non-crosslinkable. Subsequently, a second polymer containing gel, which has a viscosity that is lower than that of the first gel, is injected inside the balloon so as to coat the first gel. As with the first gel, the polymer(s) within the second gel can be, for example, natural or synthetic, biostable or biodisintegrable. However, at least one polymer within the second gel is crosslinkable. A crosslinking agent, typically of lower viscosity than the second gel, is then injected to crosslink the second gel, followed by washing with a washing solution to remove unreacted excess chemicals. If desired, an additional crosslinked layer (or more) can be formed by repeating the above injection (of the second gel), crosslinking, and washing steps. Finally, the balloon (which is biodisintegrable in this specific embodiment) is cut away from the delivery device, releasing the balloon and crosslinked polymeric body. The delivery device is then withdrawn, leaving the balloon and crosslinked polymeric body in the patient.

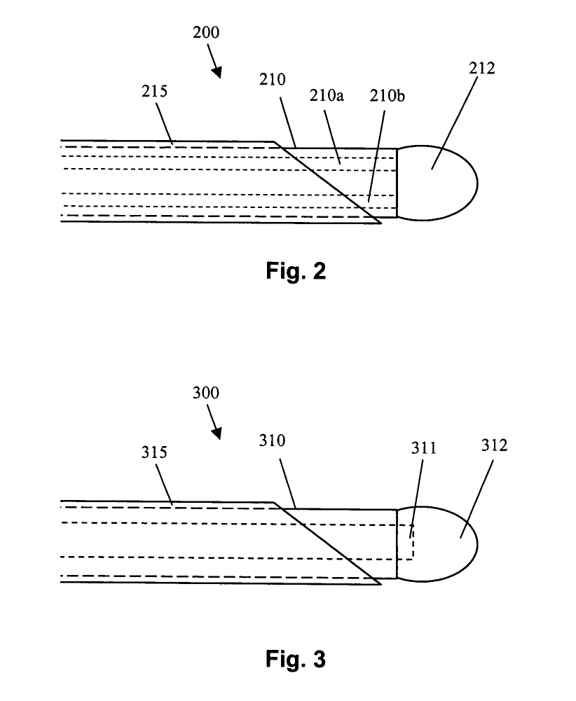

Two additional device designs are illustrated FIGS. 2 and 3. In FIG. 2, the delivery device 200 includes a shaft 210 with attached balloon 212. The shaft 210 is provided with two internal lumens 210a, 210b, which can be used to deliver polymer containing fluid and crosslinking agent, respectively, and/or can be used to simultaneously provide and withdraw washing fluid from the interior of the balloon. The shaft 210 and balloon 212 are retractably disposed within a cutting sheath 215, allowing the device to penetrate tissue.

The delivery device 300 of FIG. 3 is much like FIG. 2, as it is disposed within a cutting sheath 315 and is provided with a shaft 310 having an attached balloon 312. However, the hollow shaft 310 of FIG. 3 has one large lumen, within which is slidably disposed an inner tube 311, which can be used for the delivery of polymer containing fluid and/or crosslinking agent. The tube 311 can also be used to fill the balloon with washing fluid, in which case washing fluid can be withdrawn via the annular region between the outer wall of the tube 311 and the inner wall of hollow shaft 310 (or vice versa).

In some embodiments, the crosslinked polymeric bodies of the invention contain a therapeutic agent, which is released within the patient. In these embodiments, the crosslinked polymeric body can act both as a reservoir for the therapeutic agent and to control its release. Therapeutic agent release can be controlled in a number of ways, including varying the degree of polymer crosslinking, varying the number of crosslinked layers, linking the drug to a biodegradable polymer within the crosslinked body, and so forth.

In some embodiments, a catheter is advanced through the vasculature to form a crosslinked body at a site of interest, such as an aneurism site (where the crosslinked body acts as an aneurism filler) or an artery supplying tissue such as tumor tissue (where the crosslinked body will block blood supply to the tissue, killing the tissue and/or reducing bleeding during surgical tissue removal). Specific examples of the latter include uterine fibroid tumor embolization and liver tumor embolization.

The crosslinked polymeric bodies of the invention can also be introduced: (a) as barriers against the formation of adhesions (e.g., bands of tissue that connect anatomic sites at locations where there should not be connections, such as arteriovenous malformations (AVM's), scar tissue formation), for example, in connection with minimally invasive surgery or other post surgical procedures, (b) as scaffolding for cell growth (which scaffolding can further be provided, for example, with growth factors, cytokines, etc.), thereby supporting tissue growth and/or repair, and (c) as bulking agents (e.g., for urethral bulking)—for example, gel beads, which are only partially crosslinked and are thus readily injected, can be further crosslinked once inside the container thereby providing a crosslinked body with long-term stability.

A wide variety of polymers can be used to form the polymer containing fluids of the present invention, and can be selected from the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydoxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, polystyrenes, styrene-maleic anhydride copolymers, styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); glycosaminoglycans; polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), EPDM (ethylene propylene diene monomer) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and copolymers of the above.

The polymers may be provided in a variety of configurations, including cyclic, linear and branched configurations. Branched configurations include star-shaped configurations (e.g., configurations in which three or more chains emanate from a single branch point), comb configurations (e.g., graft polymers having a main chain and a plurality of branching side chains), and dendritic configurations (e.g., arborescent and hyperbranched polymers). The polymers can be formed from a single monomer (i.e., they can be homopolymers), or they can be formed from multiple monomers (i.e., they can be copolymers) that can be distributed, for example, randomly, in an orderly fashion (e.g., in an alternating fashion), or in blocks. In many embodiments of the present invention, biodisintegrable polymers are employed. A “biodisintegrable material” is one that, subsequent to release within the subject, undergoes dissolution, degradation, resorption and/or other disintegration processes.

Further examples of polymers for use in conjunction with the present invention, not necessarily exclusive of those listed above, and many of which are readily biodisintegrable, include: cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(ε-caprolactone-co-glycolide), carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide and poly(ethylene oxide-propylene oxide) (e.g., Pluronic acid from BASF), polyoxyethylene (polyethylene glycol), polyanhydrides, polyvinylalchol, polyethyleneamine and polypyrridine, additional salts and copolymers beyond those specifically set forth above, and blends of the forgoing.

Where biodisintegrable polymers are employed, the disintegration rate can be influenced in numerous ways, including the type and molecular weight of the polymer selected, the presence of any additives, the degree of crosslinking, and so forth.

As previously noted, at least one polymer in the polymer containing fluid(s) is chemically crosslinked. Various methods for chemically crosslinking polymers are known and include (a) covalent crosslinking, for example, with polyfunctional reagents that bridge polymer chains by reaction with functional groups along the polymer chains and (b) ionic crosslinking, for example, using polyvalent metal ions. Other crosslinking methods, such as crosslinking by exposing the polymer to light of an appropriate frequency, may also be employed.

In general, the type of crosslinking mechanism selected will be that which is sufficient to inter- and/or intra-link polymers within the polymer containing fluid, thereby forming a stable solid or semisolid material.

Chemical crosslinking is beneficial in many embodiments, because the degree of crosslinking can frequently be readily controlled, for example, by varying the type(s) of chemical crosslinking agent selected, by varying the amount(s) of crosslinking agent(s), and so forth. Chemical crosslinking agents include covalent crosslinking agents and ionic crosslinking agents. Polymers that may be covalently crosslinked do not necessarily form a mutually exclusive group from polymers that may be ionically crosslinked. Hence, polymers for use in conjunction with the present invention may be ionically crosslinked, covalently crosslinked, ionically and covalently crosslinked, or crosslinked by other methods known in the art as well.

Polyfunctional crosslinking agents are an important group of covalent crosslinking agents, and can be any compound having at least two functional groups that react with functional groups in the polymer, for example, amide or organic acid functional groups within the polymer. Any conventional polyfunctional crosslinking agent known in the art may be employed. In many embodiments, the crosslinking agent contains one or more of carboxyl, hydroxy, epoxy, halogen or amino functional groups which are capable, via well-known mechanisms such as nucleophilic or condensation reactions, of reacting with functional groups present along the polymer backbone or otherwise in the polymer structure. The polyfunctional crosslinking agent may thus comprise, for example, diazonium, azide isocyanate, acid chloride, acid anhydride, imino carbonate, amino, carboxyl, epoxy, hydroxyl, aldehyde, carbodimide and aziridine groups. Examples of crosslinking agents include polycarboxylic acids and anhydrides; polyamines; epihalohydrins; diepoxides; dialdehydes such as glutaraldehye; diols; carboxylic acid halides, ketenes and like compounds. Examples of crosslinking agents are found in U.S. Pat. Nos. 5,869,129, 5,702,754 and 6,060,534, each of which is incorporated in its entirety herein by reference.

Among the various crosslinkable polymers useful in the present invention, for example, are polymers that are characterized by the presence therein of organic acid functional groups that are reactive with polyfunctional crosslinking agents. By “organic acid functional group” is meant any organic group containing an acidic hydrogen atom such as a carboxylic, sulfonic or phosphoric acid group or a metal salt of any such acid group, particularly alkali metal salts of such acid groups such as lithium, sodium and potassium salts, and quaternary amine salts such as quaternary ammonium salts.

Crosslinking ions that are used to ionically crosslink polymers may be anions or cations, depending on whether the polymer is anionically or cationically crosslinkable. Appropriate crosslinking ions include but are not limited to polyvalent cations selected from the group consisting of calcium, magnesium, barium, strontium, boron, beryllium, aluminum, iron, copper, cobalt, lead and silver cations ions. Crosslinking anions may be selected from, but are not limited to, the group consisting of phosphate, citrate, borate, succinate, maleate, adipate and oxalate anions. More broadly, crosslinking anions are commonly derived from polybasic organic or inorganic acids. Ionic crosslinking may be carried out by methods known in the art, for example, by contacting the polymers with an aqueous solution containing dissolved ions.

Examples of ionically crosslinkable polymers, not necessarily exclusive of the various polymers listed above, which may be used in accordance with the present invention, are disclosed, for example, in U.S. Pat. Nos. 6,096,018 and 6,060,534, each of which is incorporated herein in its entirety by reference. Ionically crosslinkable polymers can be either cationic or anionic in nature and include carboxylic, sulfate, and amine functionalized polymers such as polyacrylic acid, polymethacrylic acid, polyhydroxy ethyl methacrylate, polyvinyl alcohol, polyacrylamide, poly(N-vinyl pyrrolidone), polyethylene oxide, hydrolyzed polyacrylonitrile, polyethylene amine, polysaccharides, alginic acid, pectinic acid, carboxy methyl cellulose, hyaluronic acid, heparin, heparin sulfate, chitosan, carboxymethyl chitosan, chitin, carboxymethyl starch, dextran, carboxymethyl dextran, chondroitin sulfate, cationic guar, cationic starch, alginic acid, pectinic acid, pullulan, gellan, xanthan, and collagen as well as mixtures, derivatives (such as salts and esters) and copolymers thereof. Many of these polymers are also crosslinkable using covalent crosslinking agents as well, and thus may be crosslinked with covalent crosslinking agents, with ionic crosslinking agents, or both.

In some aspects of the present invention, the polymeric crosslinked body that is formed contains one or more biologically active agents. Biologically active agents are loaded for any number of purposes including, for example, to ensure in vivo release (which may be, for example, immediate or sustained) of the biologically active agents, to influence tissue adhesion, to influence thromboresistance, to influence antihyperplastic behavior, to enhance recellularizaton, and to promote tissue neogenesis, among many other purposes.

Biologically active agents can be provided within the crosslinked polymeric body in a variety of ways, including physical admixture with polymeric species in the polymeric body, covalent attachment to polymeric species in the polymeric body, and ionic attachment to polymeric species in the polymeric body. The biologically active agent can be, for example, injected into the container admixed with the polymer containing fluid, injected bound to a polymer or other species within the polymer containing fluid, injected admixed with the crosslinking agent, or injected separately, for example, prior or subsequent to the injection of the polymer containing fluid, or prior or subsequent to the injection of the crosslinking agent.

“Biologically active agents,” “drugs,” “therapeutic agents,” “pharmaceutically active agents,” “pharmaceutically active materials,” and other related terms may be used interchangeably herein and include genetic biologically active agents, non-genetic biologically active agents and cells. Biologically active agents may be used singly or in combination.

Exemplary non-genetic biologically active agents for use in connection with the present invention include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; and (r) hormones.

Preferred non-genetic biologically active agents include paclitaxel, sirolimus, everolimus, tacrolimus, halofuginol, cladribine, dexamethasone, estradiol, ABT-578 (Abbott Laboratories), trapidil, liprostin, Actinomcin D, Resten-NG, Ap-17, abciximab, clopidogrel and Ridogrel.

Exemplary genetic biologically active agents for use in connection with the present invention include anti-sense DNA and RNA as well as DNA coding for: (a) anti-sense RNA, (b) tRNA or rRNA to replace defective or deficient endogenous molecules, (c) angiogenic and other factors including growth factors such as acidic and basic fibroblast growth factors, vascular endothelial growth factor, endothelial mitogenic growth factors, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers PVP, SP1017 (SUPRATEK), lipids such as cationic lipids, liposomes, lipoplexes, nanoparticles, or microparticles, with and without targeting sequences such as the protein transduction domain (PTD).

Cells for use in connection with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

Numerous biologically active agents, not necessarily exclusive of those listed above, have been identified as candidates for vascular treatment regimens, for example, as agents targeting restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, j) platelet aggregation inhibitors including aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (O) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional biologically active agents, not necessarily exclusive of those listed above, are also disclosed in U.S. Pat. No. 5,733,925 assigned to NeoRx Corporation, the entire disclosure of which is incorporated by reference.

In addition to the above, the crosslinked polymeric bodies of the present invention may also include one or more optional imaging contrast agents.

As with the biologically active agents above, the contrast agents can be, for example, injected into the container admixed with the polymer containing fluid, injected bound to a polymer or other species within the polymer containing fluid, injected admixed with the crosslinking agent, or injected separately, for example, prior or subsequent to the injection of the polymer containing fluid, or prior or subsequent to the injection of the crosslinking agent.

The ability to non-invasively image the regions where the formulations of the present invention (e.g., polymer containing fluid, crosslinking fluid, etc.) have been introduced (and by default where they have not been introduced) is a valuable diagnostic tool for the practice of the present invention. Among such currently available non-invasive imaging techniques are included magnetic resonance imaging (MRI), ultrasonic imaging, x-ray fluoroscopy, nuclear medicine, and others. Various categories of imaging technology have associated with them imaging contrast agents, i.e., substances that enhance the image produced by medical diagnostic equipment.

For example, x-ray based fluoroscopy is a diagnostic imaging technique that allows real-time patient monitoring of motion within a patient. To be fluoroscopically visible, formulations are typically rendered more absorptive of x-rays than the surrounding tissue. In various embodiments of the invention, this is accomplished by the use of contrast agents. Examples of contrast agents for use in connection with x-ray fluoroscopy include metals, metal salts and oxides (particularly bismuth salts and oxides), and iodinated compounds. Examples include tungsten, platinum, tantalum, iridium, gold, or other dense metal, barium sulfate, bismuth subcarbonate, bismuth trioxide, bismuth oxychloride, metrizamide, iopamidol, iothalamate sodium, iodomide sodium, and meglumine.

Ultrasound and magnetic resonance imaging can provide two- and/or three-dimensional images of a portion of the body. Ultrasound and MRI are advantageous, inter alia, because they do not expose the patient or medical practitioner to harmful radiation and can provide detailed images of the observed area. These detailed images are valuable diagnostic aids to medical practitioners and can be used to more precisely control the quantity and location of the fluid that is injected in connection with the present invention.

Magnetic resonance imaging (MRI) produces images by differentiating detectable magnetic species in the portion of the body being imaged. In the case of ¹H MRI, the detectable species are protons (hydrogen nuclei). In order to enhance the differentiation of detectable species in the area of interest from those in the surrounding environment, imaging contrast agents are often employed. These agents alter the magnetic environment of the detectable protons in the area of interest relative to that of protons in the surrounding environment and, thereby, allow for enhanced contrast and better images of the area of interest. For contrast-enhanced MRI, it is desirable that the contrast agent have a large magnetic moment, with a relatively long electronic relaxation time. Based upon these criteria, contrast agents such as Gd(III), Mn(II) and Fe(III) have been employed. Gadolinium(III) has the largest magnetic moment among these three and is, therefore, a widely-used paramagnetic species to enhance contrast in MRI. Chelates of paramagnetic ions such as Gd-DTPA (gadolinium ion chelated with the ligand diethylenetriaminepentaacetic acid) have been employed as MRI contrast agents. Chelation of the gadolinium or other paramagnetic ion is believed to reduce the toxicity of the paramagnetic metal by rendering it more biocompatible, and can assist in localizing the distribution of the contrast agent to the area of interest. Paramagnetic ion chelates can be, for example, attached to the viscosity increasing agent or they can simply be admixed with the other components of the formulation. Further information can be found, for example, in U.S. Patent Application No. 20030100830 entitled “Implantable or insertable medical devices visible under magnetic resonance imaging.”

Ultrasound uses high frequency sound waves to create an image of living tissue. A sound signal is sent out, and the reflected ultrasonic energy, or “echoes,” used to create the image. Ultrasound imaging contrast agents are materials that enhance the image produced by ultrasound equipment. Ultrasonic imaging contrast agents introduced into the formulations of the present invention can be, for example, echogenic (i.e., materials that result in an increase in the reflected ultrasonic energy upon injection of the formulation) or echolucent (i.e., materials that result in a decrease in the reflected ultrasonic energy upon injection of the formulation). Suitable ultrasonic imaging contrast agents for use in connection with the present invention include solid particles ranging from 0.01 to 50 microns in largest cross-sectional length (e.g., diameter where spherical particles are utilized), more typically 0.5 to 20 microns. Both inorganic and organic particles can be used. Examples include calcium carbonate, hydroxyapatite, silica, poly(lactic acid), poly(glycolic acid). Microbubbles can also be used as ultrasonic imaging contrast agents as is known in the imaging art. The ultrasonic imaging contrast agents for use in connection with the present invention are preferably biocompatible and stable in the formulations.

Although various embodiments are specifically illustrated and described herein, it will be appreciated that modifications and variations of the present invention are covered by the above teachings and are within the purview of the appended claims without departing from the spirit and intended scope of the invention.

1. A method of providing a polymeric body within a subject, said method comprising:
injecting a fluid comprising a crosslinkable polymer into a container that is positioned within the subject;
crosslinking said crosslinkable polymer in said container, thereby forming a crosslinked polymeric body in the container; and
releasing said crosslinked polymeric body into said subject.

2. The method of claim 1, wherein said container is an expandable container.

3. The method of claim 2, wherein said container has elastic walls.

4. The method of claim 2, wherein said container has flexible, inelastic walls.

5. The method of claim 2, wherein said expandable container is a balloon.

6. The method of claim 1, wherein said container is removed from said subject subsequent to releasing said crosslinked polymeric body.

7. The method of claim 1, wherein said container is released within said patient along with said crosslinked polymeric body.

8. The method of claim 7, wherein said container is biodisintegrable.

9. The method of claim 1, wherein said crosslinked polymeric body is biodisintegrable.

10. The method of claim 1, further comprising the step of washing the crosslinked polymeric body prior to releasing said crosslinked polymeric body.

11. The method of claim 1, wherein said crosslinked polymeric body is formed within a bodily cavity of the subject.

12. The method of claim 1, wherein said crosslinked polymeric body is formed within a bodily lumen of the subject.

13. The method of claim 1, wherein said crosslinked polymeric body is formed at a surgically created site.

14. The method of claim 1, wherein said crosslinked polymeric body is an antiadhesive body.

15. The method of claim 1, wherein said crosslinked polymeric body is formed within an aneurism.

16. The method of claim 1, wherein said crosslinked polymeric body is an embolic body.

17. The method of claim 16, wherein said embolic body is disposed in an artery of a uterine fibroid tumor.

18. The method of claim 1, wherein said crosslinked polymeric body is a bulking agent.

19. The method of claim 1, wherein said crosslinked polymeric body is a tissue scaffold.

20. The method of claim 1, wherein said crosslinked polymeric body further comprises a therapeutic agent.

21. A method of providing a polymeric body within a subject, said method comprising: (a) injecting a first fluid comprising a first polymer into a container that is positioned within the subject, (b) injecting a second fluid comprising a second, crosslinkable polymer into said container, said second fluid having a lower viscosity than said first fluid; (c) crosslinking said crosslinkable polymer in said container, thereby forming a crosslinked polymeric body in said container; and (d) releasing said crosslinked polymeric body into said subject.

22. The method of claim 21, wherein after said crosslinking step and before said releasing step, the method further comprises: (a) injecting additional fluid comprising additional crosslinkable polymer into said container; and (b) crosslinking said additional crosslinkable polymer in said container.

23. The method of claim 1, wherein after said crosslinking step and before said releasing step, the method further comprises (a) injecting additional fluid comprising additional crosslinkable polymer into said container; and (b) crosslinking said additional crosslinkable polymer in said container.

24. A crosslinked polymeric body formed by the method of claim 1, said crosslinked polymeric body conforming to the tissue contours of an injection site within a patient.

25. A device for performing the method of claim 1, said device comprising a shaft and a balloon, said shaft comprising an inner lumen for introducing said fluid into said balloon, said balloon being disposed at or proximate to a distal end of said medical device, and said medical device being adapted to release said crosslinked polymeric body within said patient.

26. The device of claim 25, wherein said shaft comprises a plurality of lumens.

27. The device of claim 25, wherein said shaft and said balloon are disposed within the lumen of an additional shaft, and wherein said additional shaft is adapted to penetrate tissue.

28. The device of claim 25, wherein said medical device is adapted to release said balloon and said crosslinked polymeric body from said device.

29. The device of claim 25, wherein said medical device is adapted to release said crosslinked polymeric body from said balloon.
